(19) United States
(12) Patent Application Publication
(10) Pub. No.: US 2012/0114753 A1
(43) Pub. Date: May 10, 2012

(54) MULTI-LAYER TABLET COMPRISING EFFERVESCENT LAYER

(75) Inventors: Hyoung-Sun YOO, Incheon (KR); Man-Ho KWON, Cheonan-si (KR); Young-Bin BANG, Asan-si (KR); Sung-Woong KIM, Asan-si (KR); Jong-Hyun LEE, Asan-si (KR); Jin-Ho YOO, Bucheon-si (KR); Bong-Haeng HUR, Cheonan-si (KR)

(73) Assignee: DASAN MEDICHEM CO., LTD., Asan-si (KR)

(21) Appl. No.: 13/289,506

(22) Filed: Nov. 4, 2011

Related U.S. Application Data
(63) Continuation-in-part of application No. PCT/KR2010/003304, filed on May 26, 2010.

(30) Foreign Application Priority Data
May 27, 2009 (KR) 10-2009-0046381

(51) Int. Cl. A61K 9/46; A61K 31/4422; A61P 9/12; A61K 31/549

(52) U.S. Cl. 424/466; 514/223.5; 514/356

(57) ABSTRACT

The present invention provides a multi-layer tablet comprising: an effervescent layer comprising hydrochlorothiazide or amlodipine or its salt as an active ingredient, a carbonate salt, and an organic acid; and a telmisartan-containing layer.

### CROSS-REFERENCE TO RELATED APPLICATIONS

This application is a continuation-in-part of PCT/KR2010/003304, filed May 26, 2010, which claims the benefit of Korean Patent Application No. 10-2009-0046381, filed May 27, 2009, the contents of each of which are incorporated herein by reference.

### FIELD OF INVENTION

The present invention relates to a multi-layer tablet comprising: an effervescent layer comprising hydrochlorothiazide or amlodipine or its salt; and a telmisartan-containing layer.

### BACKGROUND ART

Various antihypertensive agents such as diuretics, beta blockers, alpha blockers, calcium channel blockers, vasodilators, and angiotensin-receptor antagonists are used in the treatment of hypertension. In addition, pharmaceutical compositions simultaneously containing antihypertensive agents having different pharmacological mechanisms are being developed in order to obtain better treatment effects. For example, pharmaceutical formulations containing a combination of an angiotensin-receptor antagonist and a diuretic; or an angiotensin-receptor antagonist and a calcium channel blocker have been reported. One of the critical matters to be considered in designing a pharmaceutical combination composition is that respective drugs contained in the composition need to show a biological behavior similar to each single-drug composition.

As prior arts for overcoming different release characteristics of between water-insoluble telmisartan and a diuretic or a calcium channel blocker, WO 2003/059327 and WO 2006/048208 have disclosed bilayer tablets. WO 2003/059327 disclosed a bilayer pharmaceutical tablet comprising a first layer containing telmisartan in more than 90% of amorphous form in a dissolving tablet matrix, and a second layer containing hydrochlorothiazide in a disintegrating tablet matrix. In addition, WO 2006/048208 disclosed a pharmaceutical tablet comprising a first layer of telmisartan in a dissolving tablet matrix and a second layer of amlodipine in a disintegrating or eroding tablet matrix. The tablets disclosed in WO 2003/059327 and WO 2006/048208 are bilayer tablets each having a first layer formulated for immediate release of telmisartan; and a second layer formulated for immediate release of hydrochlorothiazide or amlodipine from a fast-disintegrating tablet matrix by swelling-derived disintegration.

Although the bilayer tablets disclosed in WO 2003/059327 and WO 2006/048208 are formulated so as to show immediate release of each component, they are still unsatisfactory in minimizing dissolution pattern deviations according to surrounding conditions such as gastrointestinal pH changes or gastrointestinal motility changes (for example, decreased gastrointestinal motility). That is, in case of the disintegrating tablet matrixes in which the drug-releases of diuretics or calcium channel blockers are occurred through swelling and erosion, the dissolution patterns thereof are affected according to rotating speeds of the paddle, which indicates that drug absorption may be changed according to patients' gastrointestinal motilities.

### SUMMARY OF INVENTION

### Technical Problem

The present inventors have performed various studies in order to develop a pharmaceutical combination composition comprising telmisartan together with hydrochlorothiazide or amlodipine or its salt, the composition showing immediate drug-release without being affected by environmental conditions. As a result, it was found that a multi-layer tablet having an effervescent layer comprising hydrochlorothiazide or amlodipine or its salt, a carbonate salt, and an organic acid quickly releases the drug through immediate effervescence generating CO₂ gas in the gastrointestinal tract, thereby showing uniform dissolution pattern without being affected by patients' gastrointestinal motilities.

In addition, it was also found that, when a telmisartan-containing layer is formed by formulating telmisartan through fluid bed granulation, using a fluid bed granulator that is conventionally used in a pharmaceutical field instead of a spray drier, not only can excellent dissolution characteristics of telmisartan be accomplished, but the layer can be also formed in a high yield and thus high process efficiency is expected.

Accordingly, the present invention provides a multi-layer tablet comprising: an effervescent layer comprising hydrochlorothiazide or amlodipine or its salt; and a telmisartan-containing layer.

In accordance with an aspect of the present invention, there is provided a multi-layer tablet comprising: an effervescent layer comprising hydrochlorothiazide or amlodipine or its salt as an active ingredient, a carbonate salt, and an organic acid; and a telmisartan-containing layer.

According to an embodiment of the present invention, there is provided a multi-layer tablet, wherein the active ingredient of the effervescent layer is hydrochlorothiazide, and wherein, when a dissolution test by a paddle method was performed at pH 1.2 without rotation of a paddle, the dissolution amount of hydrochlorothiazide for 0 minute to 15 minutes is 50 weight % or more of the total weight of hydrochlorothiazide.

According to another embodiment of the present invention, there is provided a multi-layer tablet, wherein the active ingredient of the effervescent layer is amlodipine or its salt, and wherein, when a dissolution test by a paddle method was performed at pH 1.2 without rotation of a paddle, the dissolution amount of amlodipine or its salt for 0 minute to 15 minutes is 50 weight % or more of the total weight of amlodipine or its salt. The salt of amlodipine may be amlodipine maleate, amlodipine besylate, amlodipine mesylate, or amlodipine camsylate.

In the multi-layer tablet of the present invention, the carbonate salt may be selected from the group consisting of sodium bicarbonate, potassium carbonate, sodium carbonate, calcium carbonate, ammonium carbonate, magnesium carbonate, and a mixture thereof; and the organic acid may be selected from the group consisting of ascorbic acid, succinic acid, tartaric acid, citric acid, malic acid, fumaric acid, and a mixture thereof.

The multi-layer tablet according to the present invention includes an effervescent layer containing hydrochlorothiazide or amlodipine or its salt, thereby quickly releasing the drug through immediate effervescence along with the generation of CO₂ gas in the gastrointestinal tract. Accordingly, the multi-layer tablet according to the present invention can minimize dissolution pattern deviations of the active ingredient, even in old patients having reduced gastrointestinal motility. That is, the multi-layer tablet according to the present invention can be expected to show uniform drug-release patterns, through minimizing any effects caused by gastrointestinal environmental changes that may result from various complications, patients' ages and states, etc. In addition, since the telmisartan-containing layer of the multi-layer tablet according to the present invention is formed by formulating telmisartan through fluid bed granulation, a high productivity as well as excellent dissolution characteristics of telmisartan can be expected.

### BRIEF DESCRIPTION OF DRAWINGS

FIG. 1 illustrates X-ray powder diffraction pattern of telmisartan Form A in the present invention.

FIG. 2 illustrates DSC (Differential Scanning calorimetry) curve of telmisartan Form A in the present invention.

FIG. 3 shows dissolution test results for telmisartan from tablets prepared according to the present invention (Examples 2, 4-1, and 7) and tablets of Comparative Example, in a buffer solution having pH 1.2.

### DETAILED DESCRIPTION OF INVENTION

The present invention provides a multi-layer tablet comprising: an effervescent layer comprising hydrochlorothiazide or amlodipine or its salt as an active ingredient, a carbonate salt, and an organic acid; and a telmisartan-containing layer.

The multi-layer tablet according to the present invention including an effervescent layer comprising hydrochlorothiazide or amlodipine or its salt quickly effervesces generating CO₂ gas in the gastrointestinal tract so as to release the drug. Accordingly, the multi-layer tablet according to the present invention can minimize dissolution pattern deviations of the active ingredient, even in old patients having reduced gastrointestinal motility. That is, when the effervescent layer is contacted with an aqueous medium, it immediately effervesces, which results in micro cavities therein. Through the micro cavities, the active ingredient is quickly dissolved. In particular, from the multi-layer tablet according to the present invention, 50% or more of hydrochlorothiazide or amlodipine are quickly dissolved in 15 minutes, even under the no-stirring condition. Such a quick dissolution is surprising, when considering that conventional formulations show 20% or less of drug release even after 120 minutes under the no-stirring condition.

Hydrochlorothiazide, whose chemical name is 6-chloro-3,4-dihydro-2H-1,2,4-benzothiadiazine-7-sulfonamide-1,1-dioxide, is a diuretic used in the treatment of an edema and hypertension and is in general orally administered. In the multi-layer tablet according to the present invention, an appropriate amount of hydrochlorothiazide may be used, based on a therapeutically effective amount thereof. For example, the amount of hydrochlorothiazide may be in the range of 5 to 50 mg per unit tablet.

Amlodipine is a calcium channel blocker and has the chemical name of 3-ethyl-5-methyl-2-(2-aminoethoxymethyl)-4-(2-chlorophenyl)-1.4-dihydro-6-methyl-3,5-pyridinedicarboxylate. The salt of amlodipine may be malate, besylate, mesylate, or camsylate salt, preferably besylate. In the multi-layer tablet according to the present invention, an appropriate amount of the amlodipine or its salt may be used, based on a therapeutically effective amount thereof. For example, the amount of the amlodipine or its salt may be in the range of 1 to 20 mg (as amlodipine) per unit tablet.

### Preparation of Multi-Layer Tablet of Telmisartan

The effervescent layer may be prepared by (a) forming a granule including hydrochlorothiazide or amlodipine or its salt as an active ingredient and a carbonate salt, (b) mixing an organic acid therewith, and then (c) compressing the obtained mixture. Alternatively, the effervescent layer may be prepared by (a′) forming a granule including the active ingredient and an organic acid, (b′) mixing a carbonate salt therewith, and then (c′) compressing the obtained mixture.

The granule may be prepared by spraying a binder solution while a mixture of hydrochlorothiazide or amlodipine or its salt, a diluent, and a carbonate salt or an organic acid is being fluidized in a fluid bed granulator. The hinder solution may be prepared by dissolving at least one binder selected from the group consisting of polyvinylpyrrolidone, polyethylene glycol, hydroxypropylmethylcellulose, and polyvinyl alcohol in water, alcohol, or a mixture thereof. Thus, the obtained granule may further include at least one binder selected from the group consisting of polyvinylpyrrolidone, polyethylene glycol, hydroxypropylmethylcellulose, and polyvinyl alcohol.

The diluent may be any diluent conventionally used in the filed of pharmaceutics. Examples of the diluent include glucose, fructose, lactose, sucrose, sorbitol, mannitol, maltol, isomaltol, xylitol, and a combination thereof. Preferably, the diluent may be isomaltol, lactose, or a mixture thereof. The amount of diluent may be in the range of 40 to 80 weight % based on the total weight of the effervescent layer, but is not limited thereto.

The carbonate salt may be any carbonate salt that reacts with an organic acid in a human body so as to generate CO₂ gas. For example, the carbonate salt may be at least one selected from the group consisting of sodium bicarbonate, potassium carbonate, sodium carbonate, calcium carbonate, ammonium carbonate, and magnesium carbonate; preferably sodium bicarbonate. In addition, the organic acid may be at least one selected from the group consisting of ascorbic acid, succinic acid, tartaric acid, citric acid, malic acid, and fumaric acid; preferably citric acid. The amount of the carbonate salt used may be in the range of 0.5 to 30 weight %, preferably 1 to 20 weight %, more preferably 1 to 15 weight %, based on the total weight of the effervescent layer. The amount of the organic acid used may be in the range of 1 to 30 weight %, preferably 1 to 15 weight %, based on the total weight of the effervescent layer.

In addition to the active ingredient (hydrochlorothiazide or amlodipine or its salt), a diluent, a carbonate salt, and an organic acid, the effervescent layer may further include pharmaceutically acceptable conventional additives, for example, a lubricant such as magnesium stearate or sodium stearyl fumarate.

Meanwhile, it is found by the present invention that a granule obtained through fluid bed granulation (that is, a granule by spraying a telmisartan-containing solution obtained by dissolving telmisartan together with meglumine and sodium hydroxide in an organic solvent onto sugars) shows excellent dissolution characteristics. In addition, since the telmisartan-containing layer can be formed in a high yield through fluid bed granulation, high process efficiency is expected.

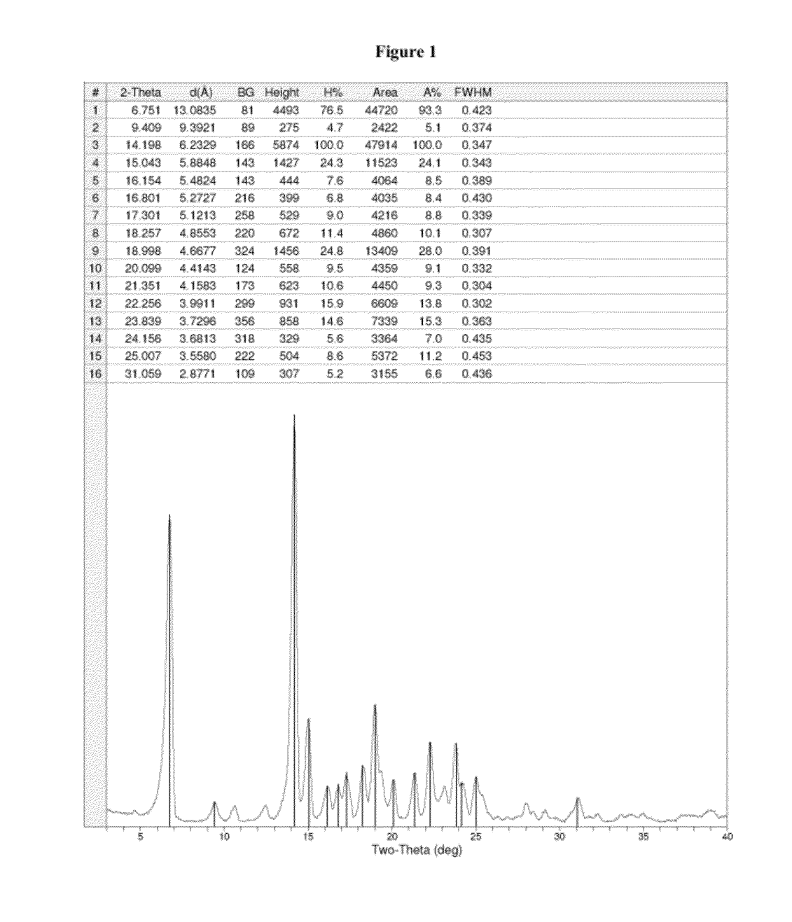

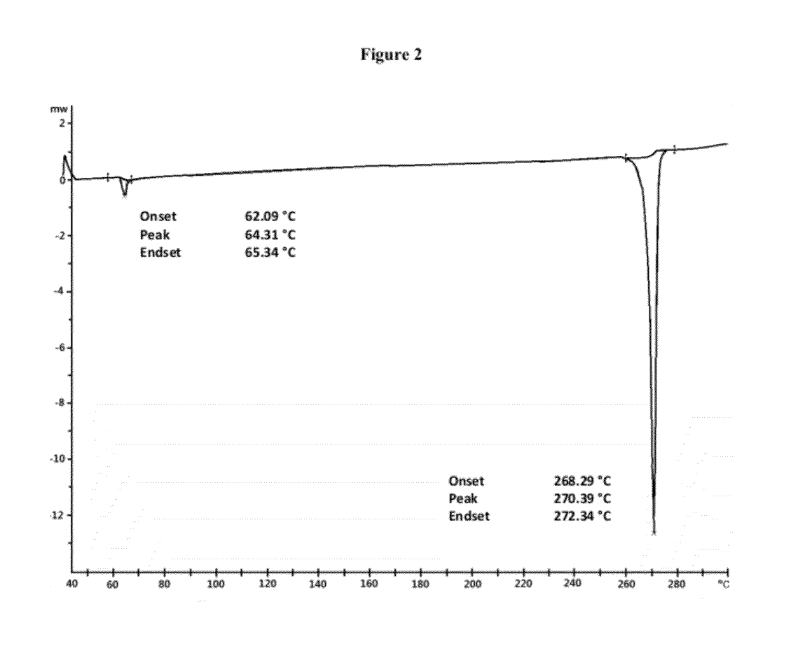

In the present invention, amorphous or crystalline Form of telmisartan is employed. In one embodiment, crystalline form of telmisartan is employed and the X-ray powder diffraction patter (XRPD) and DSC of the crystal form are provided in FIGS. 1 and 2, respectively.

The organic solvent may be anhydrous ethanol or a mixed solvent of anhydrous ethanol and methylene chloride. In the mixed solvent of anhydrous ethanol and methylene chloride, a weight ratio of anhydrous ethanol and methylene chloride may be 1:2 to 7, preferably 1:3 to 5, more preferably about 1:3. Examples of the sugars include sorbitol, mannitol, isomaltol, and etc. If necessary, the telmisartan-containing solution may further include a binder such as polyvinylpyrrolidone, hydroxypropylmethylcellulose, and polyvinyl alcohol. In the telmisartan-containing layer, an appropriate amount of telmisartan may be used, based on a therapeutically effective amount thereof, for example, in the range of 20 to 160 mg per unit tablet (e.g., a bilayer tablet). In addition, the amount of each of meglumine and sodium hydroxide may be in the range of 0.5 to 10 weight % based on unit tablet, but is not limited thereto.

According to an embodiment of the present invention, there is provided a multi-layer tablet, wherein the active ingredient of the effervescent layer is hydrochlorothiazide, and wherein, when a dissolution test by a paddle method was performed at pH 1.2 without rotation of a paddle, the dissolution amount of hydrochlorothiazide for 0 minute to 15 minutes is 50 weight % or more of the total weight of hydrochlorothiazide. According to another embodiment of the present invention, there is provided a multi-layer tablet, wherein the active ingredient of the effervescent layer is amlodipine or its salt, and wherein, when a dissolution test by a paddle method was performed at pH 1.2 without rotation of a paddle, the dissolution amount of amlodipine or its salt for 0 minute to 15 minutes is 50 weight % or more of the total weight of amlodipine or its salt. The salt of amlodipine may be amlodipine maleate, amlodipine besylate, amlodipine mesylate, or amlodipine camsylate.

In addition to the effervescent layer and the telmisartan-containing layer, the multi-layer tablet according to the present invention may further include a non-drug containing layer comprising conventionally available additives in the field of pharmaceutics, as a separate layer (e.g., it may be a triple-layer tablet form). Example of the additive may include sugars or cellulose derivatives, such as lactose, microcrystalline cellulose, isomaltol, and etc.

The multi-layer tablet according to the present invention is prepared using a conventional multi-layer tablet formation method. For example, when the multi-layer tablet is prepared in a bilayer tablet form, a mixture of an organic acid and a granule containing hydrochlorothiazide or amlodipine or its salt and a carbonate salt; or a mixture of a carbonate salt and a granule containing hydrochlorothiazide or amlodipine or its salt and an organic acid is compressed together with a lubricant such as magnesium stearate, so as to form an effervescent layer. And then, a telmisartan-containing granule or a mixture of the telmisartan-containing granule and a pharmaceutically acceptable additive (for example, diluent or lubricant) may be compressed to form a separate layer.

### EXAMPLES

Hereinafter, the present invention will be described more specifically with reference to the following examples. The following examples are only for illustrative purposes and are not intended to limit the scope of the invention.

### Example 1

### Preparation of Granules Containing Telmisartan

8.00 kg of telmisartan, 8.00 kg of polyvinylpyrrolidone, 2.40 kg of meglumine, and 0.67 kg of sodium hydroxide were dissolved in 106.00 kg of anhydrous ethanol. Granulation was performed by spraying the solution while 14.40 kg of sorbitol is being fluidized in a fluid bed granulator. The inlet temperature and the exhaust temperature of the fluid bed granulator were 60° C. and 40° C., respectively, and the spraying rate was 100.00 g per minute. Three (3) batches were prepared by using the same method above. In each batch, granules located in upper, middle, and lower portions were collected and each amount of telmisartan contained therein was measured to obtain percent (%) weights thereof relative to each theoretically calculated amount. And also, in each batch, the entire production amount compared to the entire loading amount was measured to calculate a yield. The results are shown in Table 1 and Table 2.

TABLE 1
Telmisartan in the granules (weight %)

| | Batch 1 | Batch 2 | Batch 3 |
|---|---|---|---|
| Upper portion | 98.66 | 101.28 | 100.09 |
| Middle portion | 101.89 | 99.37 | 99.55 |
| Lower portion | 98.91 | 99.74 | 99.99 |
| Average | 99.82 | 100.13 | 99.88 |

TABLE 2
Yield of each batch (weight %)

| | Batch 1 | Batch 2 | Batch 3 |
|---|---|---|---|
| Entire loading amount | 33.47 | 33.47 | 33.47 |
| Entire production amount | 33.40 | 33.20 | 33.32 |
| Yield (weight %) | 99.79 | 99.19 | 99.55 |

Referring to Table 1 and Table 2, it can be seen that, by the granulation process through fluid bed granulation, telmisartan-containing granules can be prepared in high production efficiency, while accomplishing uniformity without loss of contents.

### Example 2

### Preparation of Tablet Containing Hydrochlorothiazide and Telmisartan

0.80 kg of polyvinylpyrrolidone was completely dissolved in 10.00 kg of ethanol to obtain a binder solution. Granulation was performed by spraying the binder solution in an amount of 60.00 g per minute, while fluidizing a mixture of 1.25 kg of hydrochlorothiazide, 7.10 kg of isomaltol, 7.75 kg of anhydrous lactose, and 1.80 kg of sodium bicarbonate in a fluid bed granulator. The inlet temperature and the exhaust temperature of the fluid bed granulator were 50° C. and 30° C., respectively. 18.70 kg of the obtained granule was mixed with 1.00 kg of citric acid and 0.30 kg of magnesium stearate to obtain a mixture (mixture A).

8.00 kg of telmisartan, 8.00 kg of polyvinylpyrrolidone, 2.40 kg of meglumine, and 0.67 kg of sodium hydroxide were dissolved in 106.00 kg of anhydrous ethanol. Granulation was performed by spraying the solution while 14.40 kg of sorbitol is being fluidized in a fluid bed granulator. The inlet temperature and the exhaust temperature of the fluid bed granulator were 60° C. and 40° C., respectively, and the spraying rate was 100.00 g per minute. 33.47 kg of the obtained granule, 14.02 kg of sorbitol, and 0.51 kg of magnesium stearate were mixed to obtain a telmisartan-containing mixture (mixture B).

The mixtures A and B were compressed by using a bilayer tablet compressor (manufacturer: Gisan Machine Inc., model name: Rotary Tablet Press SPT/TP500/41) to prepare a bilayer tablet. The obtained bilayer tablet contained 12.50 mg of hydrochlorothiazide and 80.00 mg of telmisartan per unit tablet.

### Example 3

### Preparation of Tablet Containing Hydrochlorothiazide and Telmisartan

0.80 kg of polyvinylpyrrolidone was completely dissolved in 10.00 kg of ethanol to obtain a binder solution. Granulation was performed by spraying the binder solution in an amount of 60.00 g per minute, while fluidizing a mixture of 1.25 kg of hydrochlorothiazide, 7.10 kg of isomaltol, 7.75 kg of anhydrous lactose, and 1.00 kg of citric acid in a fluid bed granulator. The inlet temperature and the exhaust temperature of the fluid bed granulator were 50° C. and 30° C., respectively. 17.90 kg of the obtained granule was mixed with 1.80 kg of sodium bicarbonate and 0.30 kg of magnesium stearate to obtain a mixture (mixture A′).

The mixture A′ and the mixture B (prepared according to the same method as in Example 2) were compressed by using a bilayer tablet compressor (manufacturer: Gisan Machine Inc., model name: Rotary Tablet Press SPT/TP500/41) to prepare a bilayer tablet. The obtained bilayer tablet contained 12.50 mg of hydrochlorothiazide and 80.00 mg of telmisartan per unit tablet.

### Example 4

### Preparation of Tablet Containing Hydrochlorothiazide and Telmisartan

Using the hydrochlorothiazide-containing mixture (mixture A) and the telmisartan-containing mixture (mixture B) which were prepared according to the same manner as in Example 2, a triple-layer tablet was prepared as in Table 3. Table 3 shows the weight of each component per unit tablet. That is, a first layer containing 12.50 mg of hydrochlorothiazide was formed using mixture A, and then a second layer was formed using the mixed-powders prepared by mixing lactose, microcrystalline cellulose, or isomaltol with 0.50% of magnesium stearate (the respective powders are represented in Table 3 as lactose mixture’, ‘microcrystalline cellulose mixture’, and ‘isomaltol mixture’). And then, a third layer containing 80.00 mg of telmisartan was formed using mixture B to prepare a triple-layer tablet.

TABLE 3
Triple-layer tablet

| | Example 4-1 | | Example 4-2 | | Example 4-3 | |
|---|---|---|---|---|---|---|
| | Com- | Amt | | Amt | Com- | Amt |
| | ponent | (mg) | Component | (mg) | ponent | (mg) |
| First | Mixture A | 200 | Mixture A | 200 | Mixture A | 200 |
| layer | | | | | | |
| Second | lactose | 150 | microcrystalline | 150 | isomaltol | 150 |
| layer | mixture | | cellulose | | mixture | |
| | | | mixture | | | |
| Third | Mixture B | 480 | Mixture B | 480 | Mixture B | 480 |
| layer | | | | | | |

| | | | |
|---|---|---|---|
| Total | 830 mg | 830 mg | 830 mg |
| weight | | | |

### Example 5

### Preparation of Tablet Containing Amlodipine Besylate and Telmisartan

0.80 kg of polyvinylpyrrolidone was completely dissolved in 10.00 kg of ethanol to obtain a binder solution. Granulation was performed by spraying the binder solution in an amount of 60.00 g per minute, while fluidizing a mixture of 0.69 kg of amlodipine besylate, 7.00 kg of isomaltol, 8.32 kg of anhydrous lactose, and 1.80 kg of sodium bicarbonate in a fluid bed granulator. The inlet temperature and the exhaust temperature of the fluid bed granulator were 50° C. and 30° C., respectively. 18.61 kg of the obtained granule was mixed with 1.00 kg of citric acid and 0.30 kg of magnesium stearate to obtain a mixture (mixture C).

The mixture C and the mixture B (prepared according to the same method as in Example 2) were compressed by using a bilayer tablet compressor (manufacturer: Gisan Machine Inc., model name: Rotary Tablet Press SPT/TP50041) to prepare a bilayer tablet. The obtained bilayer tablet contained 5.00 mg of amlodipine and 80.00 mg of telmisartan per unit tablet.

### Example 6

### Preparation of Tablet Containing Amlodipine Besylate and Telmisartan

0.80 kg of polyvinylpyrrolidone was completely dissolved in 10.00 kg of ethanol to obtain a binder solution. Granulation was performed by spraying the binder solution in an amount of 60.00 g per minute, while fluidizing a mixture of 0.69 kg of amlodipine besylate, 7.00 kg of isomaltol, 8.32 kg of anhydrous lactose, and 1.00 kg of citric acid in a fluid bed granulator. The inlet temperature and the exhaust temperature of the fluid bed granulator were 50° C. and 30° C., respectively. 17.81 kg of the obtained granule was mixed with 1.80 kg of sodium bicarbonate and 0.30 kg of magnesium stearate to obtain a mixture (mixture C′).

The mixture C′ and the mixture B (prepared according to the same method as in Example 2) were compressed by using a bilayer tablet compressor (manufacturer: Gisan Machine Inc., model name: Rotary Tablet Press SPT/TP500/41) to prepare a bilayer tablet. The obtained bilayer tablet contained 5.00 mg of amlodipine and 80.00 mg of telmisartan per unit tablet.

### Example 7

### Preparation of Tablet Containing Hydrochlorothiazide and Telmisartan

240.00 g of telmisartan, 72.00 g of polyvinylpyrrolidone, 72.00 g of meglumine, and 20.16 g of sodium hydroxide were dissolved in 3200.00 g of anhydrous ethanol. Granulation was performed by spraying the solution while 600.00 g of isomaltol is being fluidized in a fluid bed granulator. The inlet temperature and the exhaust temperature of the fluid bed granulator were 60° C. and 40° C., respectively, and the spraying rate was 6 g per minute. 334.72 kg of the obtained granule, 140.27 g of isomaltol, and 5.01 g of magnesium stearate were mixed to obtain a telmisartan-containing mixture.

Using the obtained telmisartan-containing mixture and the hydrochlorothiazide-containing mixture prepared according to the same method as in Example 2 (that is, mixture A), a bilayer tablet was prepared according to the same manner as in Example 2. The obtained bilayer tablet contained 12.5 mg of hydrochlorothiazide and 80.00 mg of telmisartan per unit tablet.

### Example 8

### Dissolution Test

Dissolution tests and analytical methods are as follows.

(1) Dissolution Test

A dissolution test was performed by using 900 mL of a buffer solution having pH 1.2 (Disintegration Test Solution 1 in General Test Methods of Korean Pharmacopoeia 9th Revision) as a dissolution solution at a temperature of 37° C., according to the Dissolution Test Method II (paddle method) in General Test Methods of Korean Pharmacopoeia 9th Revision. After 15 minutes, 5 mL of the dissolution solution for each sample was collected and then filtered through a membrane filter having a pore of 0.45 The filtrate was used to measure a dissolution rate.

(2-1) Analytical Method for Hydrochlorothiazide or Telmisartan
Column: ACQUITY HPLC® BEH C18 1.7 um 2.1×50 mm
Mobile phase

A: a solution prepared by adding phosphoric acid to 1000 ml of 0.05 mol/L potassium dihydrogen phosphate solution until the acidity thereof attains pH 3.0.

B: acetonitrile

TABLE 4
Dissolution Result

| Time | Flow rate | Mobile phase A | Mobile phase B |
|---|---|---|---|
| (min.) | (mL/min.) | (%) | (%) |
| 0 | 0.4 | 80 | 20 |
| 0.8 | 0.4 | 80 | 20 |
| 1.0 | 0.4 | 63 | 37 |
| 3.5 | 0.4 | 63 | 37 |
| 4.0 | 0.4 | 80 | 20 |
| 5.0 | 0.4 | 80 | 20 |

Detector: UV-spectrophotometer (271 nm)
Column temperature: 40° C.

(2-2) Analytical Method for Amlodipine
Column: ACQUITY HPLC® BEH C18 1.7 um 2.1×100 mm
Mobile phase: a mixed solution of methanol and 0.03 mol/L potassium dihydrogen phosphate solution (60:40)
Flow rate: 0.35 mL/min.
Detector: UV-spectrophotometer (237 nm)
Column temperature: 40° C.

### Example 9

### Dissolution Test of the Tablet Containing Hydrochlorothiazide and Telmisartan

A dissolution test was performed on the tablets prepared according to Examples 2 and 3 (containing 12.5 mg of hydrochlorothiazide and 80 mg of telmisartan). Using commercially available Micardis Plus™ tablet as a comparative example, a dissolution test was also performed thereon. The dissolution tests were performed on the 6 tablets for each). In order to identify an effect according to stirring rates of the paddle, a rotation speed of the paddle was set to 0, 25 and 50 rpm and dissolution rates according to rotation speeds was measured for each tablet. When the rotation speed was 0 rpm, the dissolution rate was measured after 15, 30, 45, 60, 90, and 120 minutes. Dissolution rates (%) of hydrochlorothiazide according to rotation speeds of the paddle are shown in Table 5 and Table 6.

TABLE 5
Dissolution rates of hydrochlorothiazide according to rotation speeds of
the paddle (pH 1.2, 15 minutes)

| Rotation speed (rpm) | Tablet | Dissolution rate (%) |
|---|---|---|
| 50 | Example 2 | 100.6 ± 2.1 |
| | Example 3 | 96.5 ± 1.7 |
| | Comparative Example | 94.5 ± 0.7 |
| 25 | Example 2 | 92.7 ± 1.5 |
| | Example 3 | 91.1 ± 2.8 |
| | Comparative Example | 50.2 ± 11.7 |
| 0 | Example 2 | 79.9 ± 7.0 |
| | Example 3 | 75.1 ± 11.8 |
| | Comparative Example | 4.6 ± 2.6 |

TABLE 6
Dissolution rates of hydrochlorothiazide at 0 rpm (pH 1.2)

| | 15 min. | 30 min. | 45 min. | 60 min. | 90 min. | 120 min. |
|---|---|---|---|---|---|---|
| Example 2 | 79.9 ± 7.0 | 84.0 ± 9.4 | 84.6 ± 8.3 | 85.2 ± 7.6 | 86.4 ± 7.7 | 87.5 ± 7.3 |
| Comparative | 4.6 ± 2.6 | 7.9 ± 5.5 | 8.5 ± 3.9 | 10.1 ± 4.5 | 13.2 ± 5.4 | 15.4 ± 5.9 |
| Example | | | | | | |

Referring to Table 5, it can be seen that tablets according to the present invention show a dissolution rate of 50 weight % or more within 15 minutes independently of the rotation speeds. In particular, as shown in Table 6, the tablets of Comparative Example show a dissolution rate of 20% or less even after 2 hours, on the other hand, the tablets according to the present invention show a dissolution rate of 50% or more within 15 minutes. Thus, it can be expected that an immediate drug release may be always expected independently of surrounding environments, especially in old patients having decreased gastrointestinal motility.

### Example 10

### Dissolution Test of the Tablet Containing Amlodipine Besylate and Telmisartan

A dissolution test was performed on the tablets prepared according to Examples 5 and 6 (containing 5.00 mg of amlodipine) and 80 mg of telmisartan). Using commercially available Norvasc™ 5.00 mg tablet (Pfizer Inc.) as a comparative example, a dissolution test was also performed thereon. The dissolution tests were performed on the 6 tablets for each. In order to identify an effect according to stirring rates of the paddle, a rotation speed of the paddle was set to 0 and 50 rpm and dissolution rates according to rotation speeds was measured for each tablet. Dissolution rates (%) of amlodipine besylate according to rotation speeds of the paddle are shown in Table 7 and Table 8.

TABLE 7
Dissolution rates of amlodipine besylate at 50 rpm (pH 1.2)

| | Dissolution rates (%) |
|---|---|
| Example 5 | 88.5 ± 5.2 |
| Example 6 | 82.5 ± 3.7 |
| Comparative example | 85.6 ± 4.1 |

TABLE 8
Dissolution rates of amlodipine besylate at 0 rpm (pH 1.2)

| | Dissolution rates (%) |
|---|---|
| Example 5 | 62.9 ± 6 |
| Example 6 | 58.5 ± 2.5 |
| Comparative example | 2.1 ± 1.3 |

Referring to Tables 7 and 8, it can be seen that the bilayer tablets according to the present invention have a dissolution rate of 50 weight % or more within 15 minutes. In particular, when the rotation speed of the paddle is 0 rpm, the dissolution rates of the bilayer tablets according to the present invention were much higher than those of the tablets of Comparative Example.

### Example 11

### Dissolution Test of the Triple-Layer Tablet Containing Hydrochlorothiazide and Telmisartan

A dissolution test was performed on the tablets prepared according to Example 4 (containing 12.5 mg of hydrochlorothiazide and 80 mg of telmisartan). The dissolution tests were performed on the 6 tablets for each. In order to identify an effect according to stirring rates of the paddle, a rotation speed of the paddle was set to 0 and 50 rpm and dissolution rates according to rotation speeds was measured for each tablet. Dissolution rates (%) of hydrochlorothiazide according to rotation speeds of the paddle are shown in Table 9.

TABLE 9
Dissolution rates of hydrochlorothiazide according to rotation speeds of
the paddle

| Rotation speed (rpm) | Tablets | Dissolution rates (%) |
|---|---|---|
| 50 | Example 4-1 | 95.3 ± 2.8 |
| | Example 4-2 | 98.5 ± 3.1 |
| | Example 4-3 | 96.9 ± 2.2 |
| 0 | Example 4-1 | 68.7 ± 5.7 |
| | Example 4-2 | 64.5 ± 7.8 |
| | Example 4-3 | 66.3 ± 2.2 |

### Example 12

### Dissolution Test on Hydrochlorothiazide and Telmisartan-Containing Tablet

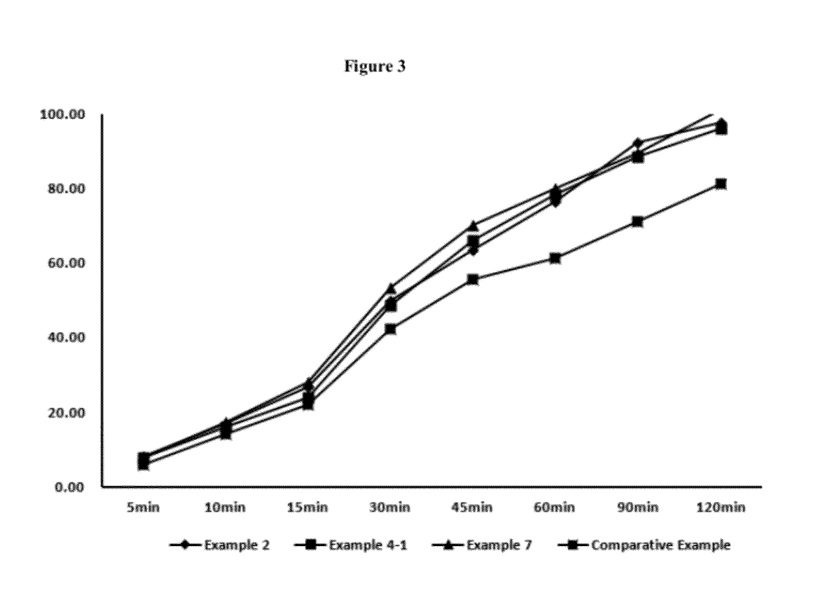

A dissolution test of telmisartan was performed on the tablets prepared according to Example 2, 4-1, and 7. Using commercially available Micardis Plus™ tablet as a comparative example, a dissolution test was also performed thereon. The dissolution tests were performed on the 3 tablets for each at 50 rpm of the paddle rotation speed. For the dissolution test of telmisartan, 5 mL of the dissolution solution was collected 15, 30, 45, 60, 90, and 120 minutes after this experiment began, and then filtered through a membrane filter having a pore of 0.45 μm. The filtrate was used to measure a dissolution rate of telmisartan. Dissolution test results are shown in FIG. 3. Referring to FIG. 3, it can be seen that the tablets according to the present invention show a telmisartan-dissolution profile equivalent to or better than that of Comparative Example.

We claim:

1. A multi-layer tablet comprising:
an effervescent layer comprising hydrochlorothiazide or amlodipine or its salt as an active ingredient, a carbonate salt, and an organic acid; and
a telmisartan-containing layer.

2. The multi-layer tablet of claim 1, wherein the active ingredient of the effervescent layer is hydrochlorothiazide, and wherein, when a dissolution test by a paddle method was performed at pH 1.2 without rotation of a paddle, the dissolution amount of hydrochlorothiazide for 0 minute to 15 minutes is 50 weight % or more of the total weight of hydrochlorothiazide.

3. The multi-layer tablet of claim 1, wherein the active ingredient of the effervescent layer is amlodipine or its salt, and wherein, when a dissolution test by a paddle method was performed at pH 1.2 without rotation of a paddle, the dissolution amount of amlodipine or its salt for 0 minute to 15 minutes is 50 weight % or more of the total weight of amlodipine or its salt.

4. The multi-layer tablet of claim 3, wherein the salt of amlodipine is amlodipine maleate, amlodipine besylate, amlodipine mesylate, or amlodipine camsylate.

5. The multi-layer tablet of claim 1, wherein the carbonate salt is selected from the group consisting of sodium bicarbonate, potassium carbonate, sodium carbonate, calcium carbonate, ammonium carbonate, magnesium carbonate, and a mixture thereof.

6. The multi-layer tablet of claim 1, wherein the organic acid is selected from the group consisting of ascorbic acid, succinic acid, tartaric acid, citric acid, malic acid, fumaric acid, and a mixture thereof.
